(19) United States
(12) Patent Application Publication
(10) Pub. No.: US 2026/0130681 A1
(43) Pub. Date: May 14, 2026

(54) SEPARABLE STONE-REMOVAL APPARATUS

(71) Applicant: YULIN WANG, SHENZHEN (CN)

(72) Inventor: YULIN WANG, SHENZHEN (CN)

(21) Appl. No.: 18/959,836

(22) Filed: Nov. 26, 2024

(30) Foreign Application Priority Data
Nov. 12, 2024 (CN) 2024116052870

(51) Int. Cl. A61B 17/221; A61B 17/22

(52) U.S. Cl. CPC A61B 17/221; A61B 2017/22079

(57) ABSTRACT

A separable stone-removal apparatus is provided, which includes a housing, with a negative pressure unit fixedly mounted inside the housing, a display panel for display fixedly mounted on the upper end of the housing, a circuit board for adjusting the opening and closing state of the negative pressure unit and the display state of the display panel fixedly mounted inside the housing, and an energy storage unit fixedly mounted inside the housing. The energy storage unit is used to provide power for the circuit board, the negative pressure unit, and the display panel. The separable stone-removal apparatus utilizes negative pressure to suck small stones out of tonsillar crypts, and transfers the stones to a storage chamber for temporary storage, thereby realizing a simple removal operation of small stones.

### TECHNICAL FIELD

The present disclosure relates to the technical field of stone absorption, and in particular to a separable stone-removal apparatus.

### BACKGROUND

Tonsil stones predominantly develop within the crypts of the tonsils, which can become obstructed over extended periods, leading to inadequate drainage of secretions. This stagnation results in the accumulation of caseous materials, which subsequently facilitates the deposition of insoluble inorganic salts, such as calcium and magnesium, forming stones. The core of these tonsil stones may consist of small foreign bodies, desquamated epithelial cells, or bacteria. Although tonsil stones are relatively small, they can obstruct the glandular ducts or crypts of the tonsils, resulting in impaired drainage, bacterial proliferation, and the accumulation of food debris. This condition is often associated with halitosis characterized by a foul odor reminiscent of rotten eggs, which can significantly diminish the patient's quality of life. Common clinical manifestations arising from the prolonged presence of tonsil stones, particularly when not addressed promptly, include purulent infections of the inner wall of the tonsil crypts surrounding the stones. Patients may experience symptoms such as sore throat and referred ear pain. In more severe instances, individuals may encounter difficulties in swallowing, slurred speech, increased salivation, and challenges in mouth opening.

Small stones are present within the crypts and fissures of the tonsils, and their removal poses significant challenges. The prevalent techniques employed for the extraction of these small stones typically involve the assistance of external agents utilizing tools such as scrapers or medical forceps, or the application of negative pressure to aspirate the stones from the tonsillar crypts. However, these methods of manual extraction are not only difficult to visualize but also present challenges in terms of grip, as the small stones are prone to dislodgment and may inadvertently be swallowed due to involuntary hand movements.

In contemporary society, the removal of tonsil stones necessitates that individuals utilize tweezers or a relatively long cotton swab. This process involves positioning oneself in front of a mirror, illuminating the area with a light source, and subsequently, with the aid of both the light and the mirror, carefully extracting the larger tonsil stones from the crypts or crevices. However, this technique is often cumbersome and poses a risk of damaging the tonsils, which can lead to complications in daily life.

### SUMMARY

In order to solve the problem mentioned in the background art, a separable stone-removal apparatus is provided.

To solve the technical problems above, the present disclosure provides a separable stone-removal apparatus, which includes:
a housing, with a negative pressure unit fixedly mounted inside the housing, a display panel for displaying fixedly mounted on the upper end of the housing, a circuit board for adjusting the opening and closing state of the negative pressure unit and the display state of the display panel fixedly mounted inside the housing, and an energy storage unit fixedly mounted inside the housing. The energy storage unit is used to provide power for the circuit board, the negative pressure unit, and the display panel, and the energy storage unit is a rechargeable lithium battery. The display panel has a touch function, and the circuit board can be controlled by the display panel with a touch function, or the upper end face of the housing is also embedded with a control button for controlling the circuit board.

The separable stone-removal apparatus further includes: a separable handle unit, suitable for stone removal operations under different working conditions, and the separable handle unit is arranged on the outer side of the housing.

Preferably, the negative pressure unit is a negative pressure suction pump, and the separable handle unit includes a connecting member mounted inside the housing. A handle is mounted on the side of the connecting member away from the housing, and the handle is hollow. A first suction assembly or a second suction assembly is provided on the end of the handle away from the housing, such that the first suction assembly or the second suction assembly can be selectively assembled with the handle according to different working conditions.

Preferably, the first suction assembly includes a first base fixedly sleeved on an end of the handle away from the housing, and the center of the first base is in the shape of an opening. A docking column is fixedly mounted at the opening of the first base, a first connecting handle is slidably mounted on the outer surface of the first base, and the first connecting handle and the first base are snap-fitted. The first connecting handle and the internal space of the first base form a storage chamber, and the storage chamber is used for temporary storage of stones. A communicating hole is provided at the end of the first connecting handle away from the handle, and a docking component is also provided on the end face of the first connecting handle away from the handle. A protective component is also provided inside the storage chamber, the stones extracted by the docking component will remain inside the storage chamber, and the protective component prevents the stones from blocking the port of the docking column.

Preferably, the docking component includes a first suction head or a second suction head. A suction flow channel is provided inside the first suction head, and a straight groove channel is provided inside the second suction head. The inner diameter of the straight groove channel is less than the inner diameter of the suction flow channel, and the outer diameter of the second suction head is less than the outer diameter of the first suction head. A connecting head is fixedly mounted between the first suction head or the second suction head and the communicating hole, and the connecting head serves to communicate the communicating hole with the suction flow channel or the straight groove channel. A flaring groove is also provided at one end of the first suction head away from the first connecting handle, and the flaring groove is concentrically communicated with the suction flow channel. The flaring groove can be used to assemble expansion components such as a straight pipe or an elbow pipe.

Preferably, the protective component includes an isolation cover sleeved on the outer surface of the docking column, the isolation cover is in a closed shape at one end thereof away from the docking column, and the outer surface of the isolation cover is provided with an air hole for ventilation, such that stones can be prevented from directly entering the holes of the docking column.

Preferably, the second suction assembly includes a second base fixedly arranged on an end face of the handle away from the housing, and the other end of the second base is also sleeved with an adapter seat block. The outer surface of the adapter seat block is also sleeved with a second connecting handle, and the second base and the adapter seat block, as well as the second connecting handle and the adapter seat block, are all snap-fitted. The interior of the second connecting handle, the center of the second base, and the center of the adapter seat block are all hollow. The end of the second connecting handle away from the handle is fixedly connected and mounted with a suction pipe, and the end of the second connecting handle away from the handle is also provided with a visualization component. The second connecting handle is tapered as a whole, with one end close to the handle being larger and the other end being smaller.

Preferably, the end of the second connecting handle away from the handle is elliptical, the visualization component includes an LED light and a second camera respectively fixedly mounted at two focal points of two ends of the elliptical second connecting handle, and the suction pipe is located between the LED light and the second camera. The LED light and the second camera are both controlled by the circuit board, and the LED light can provide lighting for the second camera.

Preferably, the end of the second connecting handle away from the handle is in the shape of a rounded rectangle, the visualization component includes a first camera fixedly mounted on the end of the second connecting handle away from the handle, and the first camera and the suction pipe are oppositely distributed on the rounded rectangular end face of the second connecting handle. The first camera is controlled by the circuit board, and the first camera can capture details inside the user's mouth and display them on the display panel.

Preferably, the interior of the flaring groove and the exterior of the suction pipe are mounted with a straight pipe or an elbow pipe. Based on the positioning of the stone within the user's throat, the operator can selectively utilize either the straight pipe or the elbow pipe.

Preferably, an end face of the first connecting handle away from the housing is eccentrically arranged with respect to the communicating hole, and the suction flow channel is curved. The interval between the eccentrically arranged connecting hole and the bottom of the storage chamber is increased. The entire stone-removal apparatus is tilted downward during operation, and the stones entering the storage chamber will accumulate at the inclined bottom end of the storage chamber.

Preferably, the protective component includes an eccentric sleeve ring fixedly mounted inside the first connecting handle, and the eccentric sleeve ring and the first connecting handle are eccentrically arranged. A portion of the eccentric sleeve ring extending into the first connecting handle is a notch portion. An annular water-absorbing and breathable filter element is provided inside the eccentric sleeve ring, and the port of the docking column away from the housing abuts against the surface of the water-absorbing and breathable filter element. A driving member is also arranged between the first connecting handle and the eccentric sleeve ring, the water-absorbing and breathable filter element has a cotton outer coating with built-in activated carbon particles, which can prevent saliva in the oral cavity from entering the interior of the docking column during suction.

Preferably, the driving member includes an electric motor fixedly mounted on the outer surface of the first connecting handle. The output end of the electric motor is rotatably embedded in the interior of the eccentric sleeve ring, and the other end of the electric motor is fixedly connected to a rechargeable power supply. The surface of the rechargeable power supply is also provided with a charging port for charging. The inner and outer surfaces of the water-absorbing and breathable filter element are respectively fixedly mounted with a fixing ring and a gear ring, and the fixing ring and the gear ring are both slidably mounted in the interior of the eccentric sleeve ring. The outer portion of the gear ring is engaged with a first gear, and the first gear is axially slidably sleeved on the outer portion of the output end of the electric motor. The rechargeable power supply provides power for the electric motor during operation.

Preferably, the combination structure of the first connecting handle and the eccentric sleeve ring includes a first separation part and a second separation part, and the first separation part and the second separation part are assembled with screws, that is, the first separation part and the second separation part can be disassembled and assembled, such that the water-absorbing and breathable filter element can be conveniently replaced.

Preferably, the electric motor is arranged on the outer surface of the first separation part, and the overall volume of the first separation part is less than that of the second separation part. Therefore, after the electric motor is assembled, the obstruction of the operator's line of sight can be minimized.

Preferably, an inner gear ring is rotatably provided at one end face of the interior of the storage chamber away from the water-absorbing and breathable filter element. A second gear and a third gear are respectively engaged with the inner wall of the inner gear ring and the outer wall of the gear ring, and a rotating shaft is concentrically fixedly connected between the second gear and the third gear. The rotating shaft is rotatably mounted to the interior of the storage chamber through a bracket. A plurality of extension strips equidistantly distributed on the circumference of the inner gear ring are fixedly provided at one end face thereof close to the water-absorbing and breathable filter element, and each extension strip is slidably fitted with the inner wall of the storage chamber. The inner gear ring can rotate synchronously with the gear ring, and the accumulated stones are moved flat by the extension strips.

Preferably, the outer surfaces of the electric motor and the rechargeable power supply are both fixedly covered with a waterproof outer covering to ensure the waterproof performance of the electric motor and the rechargeable power supply.

Preferably, the connecting member includes a sleeve pipe, two ends of which are fixedly mounted to the housing and the handle respectively. An air pipe and a data transmission line are provided inside the sleeve pipe, and two ends of the air pipe are connected and docked with the suction end of the negative pressure suction pump and the handle respectively.

Compared with the prior art, the stone-removal apparatus of the present disclosure has the following beneficial effects.

The stone-removal apparatus employs negative pressure to extract small stones from the tonsillar crypts and subsequently transfers these stones to a designated storage chamber for temporary containment, thereby facilitating a straightforward removal process.

Additionally, the stone-removal apparatus is equipped with a camera and lighting functionalities. The camera captures images and relays them to a display panel for visual feedback. By illuminating the small tonsil stones, the stone-removal apparatus enhances the ease of removal, thereby offering practicality and convenience for personal hygiene in relation to small tonsil stones. Furthermore, the compact design of the product allows for portability and versatility in its application across various settings.

### BRIEF DESCRIPTION OF DRAWINGS

FIG. 1 is a schematic diagram of the structure of the stone-removal apparatus according to a first and second embodiments of the present disclosure.

FIG. 2 is a schematic diagram of the structure of the first and second suction heads according to the first embodiment of the present disclosure.

FIG. 3 is a schematic diagram of the structure of the isolation cover according to the first embodiment of the present disclosure.

FIG. 4 is a schematic diagram of the structure of the communicating hole and the suction flow channel according to the first embodiment of the present disclosure.

FIG. 5 is a schematic diagram of the structure of the electric motor and the rechargeable power supply according to a third embodiment of the present disclosure.

FIG. 6 is a schematic diagram of the internal structure of the eccentric sleeve ring according to the third embodiment of the present disclosure.

FIG. 7 is a schematic diagram of the position distribution structure of the water-absorbing and breathable filter element and the docking column according to the third embodiment of the present disclosure.

FIG. 8 is a schematic diagram of the structure of the inner gear ring and the extension strip according to the third embodiment of the present disclosure.

FIG. 9 is a schematic diagram of the engaging state structure of the second gear and the inner gear ring according to the third embodiment of the present disclosure.

FIG. 10 is a schematic diagram of the eccentric distribution structure of the communicating hole according to the third embodiment of the present disclosure.

In the drawings: 1. housing; 2. display panel; 3. control button; 4. sleeve pipe; 5. handle; 6. first connecting handle; 7. second connecting handle; 8. first base; 9. second base; 10. adapter seat block; 11. suction pipe; 12. LED light; 13. first camera; 14. straight pipe; 15. elbow pipe; 16. first suction head; 17. second suction head; 18. flaring groove; 19. connecting head; 20. docking column; 21. isolation cover; 22. air hole; 23. second camera; 24. eccentric sleeve ring; 25. gear ring; 26. first gear; 27. electric motor; 28. rechargeable power supply; 29. charging port; 30. first separation part; 31. second separation part; 32. fixing ring; 33. water-absorbing and breathable filter element; 34. inner gear ring; 35. extension strip; 36. second gear; 37. third gear; 38. rotating shaft; 39. communicating hole; 40. suction flow channel.

### DETAILED DESCRIPTION

The technical solutions in the embodiments of the present disclosure will be clearly and completely described below with reference to the accompanying drawings in the embodiments of the present disclosure. Obviously, the described embodiments are part of the embodiments of the present disclosure, rather than all of the embodiments. Based on the embodiments in the present disclosure, all other embodiments obtained by those of ordinary skill in the art without creative efforts fall within the scope of protection of the present disclosure.

### Embodiment 1

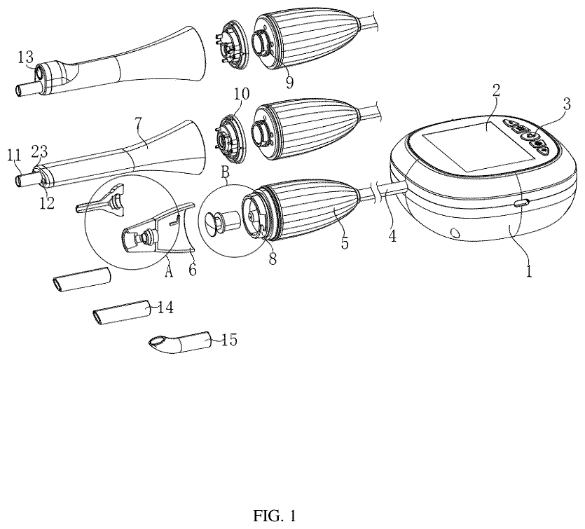

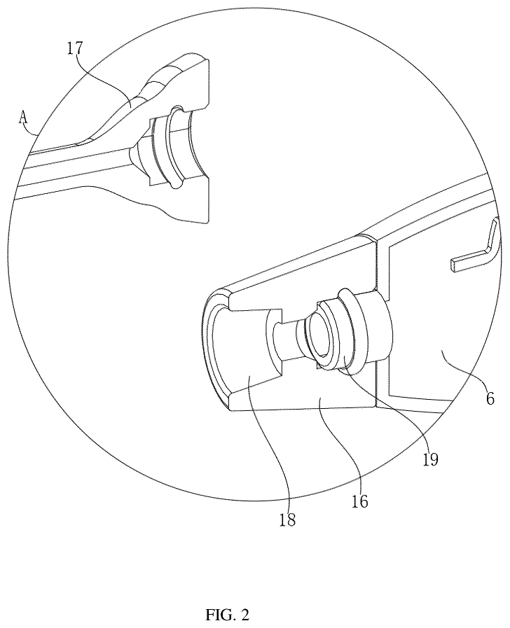

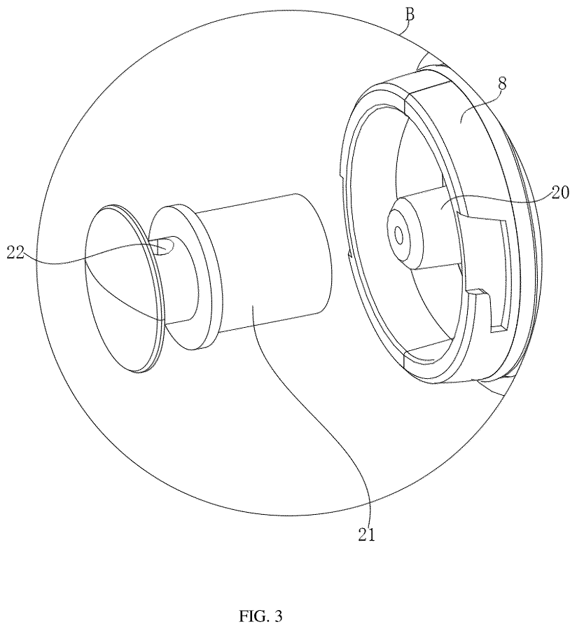

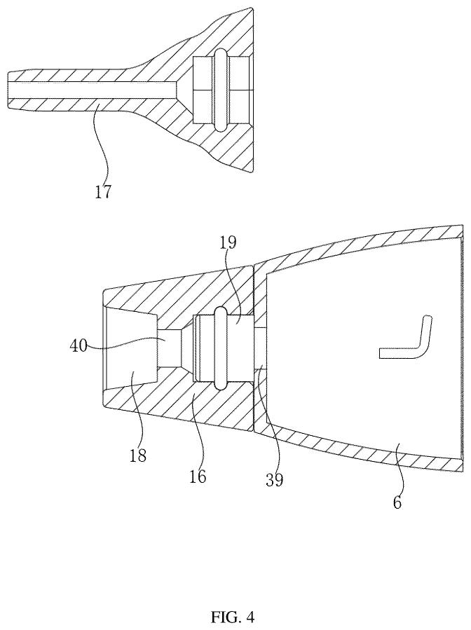

Please refer to FIGS. 1 to 4. A separable stone-removal apparatus is provided, which includes:
a housing 1, with a negative pressure unit fixedly mounted inside the housing 1, a display panel 2 for display fixedly mounted on the upper end of the housing 1, a circuit board for adjusting the opening and closing state of the negative pressure unit and the display state of the display panel 2 fixedly mounted inside the housing 1, and an energy storage unit fixedly mounted inside the housing 1. The energy storage unit is used to provide power for the circuit board, the negative pressure unit, and the display panel 2, and the energy storage unit is a rechargeable lithium battery. The display panel 2 has a touch function, and the circuit board can be controlled by the display panel with a touch function, or the upper end face of the housing is also embedded with a control button 3 for controlling the circuit board.

The separable stone-removal apparatus further includes: a separable handle unit, suitable for stone removal operations under different working conditions, and the separable handle unit is arranged on the outer side of the housing 1.

The negative pressure unit is a negative pressure suction pump, and the separable handle unit includes a connecting member mounted inside the housing 1. A handle 5 is mounted on the side of the connecting member away from the housing 1, and the handle 5 is hollow. A first suction assembly or a second suction assembly is provided on the end of the handle 5 away from the housing 1, such that the first suction assembly or the second suction assembly can be selectively assembled with the handle 5 according to different working conditions.

The first suction assembly includes a first base 8 fixedly sleeved on an end of the handle 5 away from the housing 1, and the center of the first base 8 is in the shape of an opening. A docking column 20 is fixedly mounted at the opening of the first base 8, a first connecting handle 6 is slidably mounted on the outer surface of the first base 8, and the first connecting handle 6 and the first base 8 are snap-fitted. The first connecting handle 6 and the internal space of the first base 8 form a storage chamber, and the storage chamber is used for temporary storage of stones. A communicating hole 39 is provided at the end of the first connecting handle 6 away from the handle 5, and a docking component is also provided on the end face of the first connecting handle away from the handle. A protective component is also provided inside the storage chamber, the stones extracted by the docking component will remain inside the storage chamber, and the protective component prevents the stones from blocking the port of the docking column 20.

The docking component includes a first suction head 16 or a second suction head 17. A suction flow channel 40 is provided inside the first suction head 16, and a straight groove channel is provided inside the second suction head 17. The inner diameter of the straight groove channel is less than the inner diameter of the suction flow channel 40, and the outer diameter of the second suction head 17 is less than the outer diameter of the first suction head 16. A connecting head 19 is fixedly mounted between the first suction head 16 or the second suction head 17 and the communicating hole 39, and the connecting head 19 serves to communicate the communicating hole 39 with the suction flow channel 40 or the straight groove channel. A flaring groove 18 is also provided at one end of the first suction head 16 away from the first connecting handle 6, and the flaring groove 18 is concentrically communicated with the suction flow channel 40. The flaring groove 18 can be used to assemble expansion components such as a straight pipe 14 or an elbow pipe 15.

The protective component includes an isolation cover 21 sleeved on the outer surface of the docking column 20, the isolation cover 21 is in a closed shape at one end thereof away from the docking column 20, and the outer surface of the isolation cover 21 is provided with air holes 22 for ventilation, such that stones can be prevented from directly entering the holes of the docking column 20 due to the presence of the isolation cover 21.

The interior of the flaring groove 18 and the exterior of the suction pipe 11 are mounted with a straight pipe 14 or an elbow pipe 15. Based on the positioning of the stone within the user's throat, the operator can selectively utilize either the straight pipe 14 or the elbow pipe 15.

Operational principle: The operator can selectively attach either the first suction head 16 or the second suction head 17 to the distal end of the first connecting handle 6, depending on the location and dimensions of the tonsil stones. Additionally, the second suction head 17 can be connected to either the straight pipe 14 or the elbow pipe 15 via the flaring groove 18.

The operator then extends the distal end of the first connecting handle 6 away from the housing 1 into the oral cavity. Prior to this extension, the negative pressure suction pump located within the housing 1 is activated, generating negative pressure. Once the distal end of the second suction head 17, or the ends of the straight pipe 14 and the elbow pipe 15, make contact with the stones, the negative pressure facilitates the direct suction of the stones. Subsequently, the stones are transported to the storage chamber through the action of suction, thereby achieving the efficient removal of tonsil stones.

### Embodiment 2

Please refer to FIG. 1. This embodiment further illustrates embodiment 1. The second suction assembly includes a second base 9 fixedly arranged on an end face of the handle 5 away from the housing 1, and the other end of the second base 9 is also sleeved with an adapter seat block 10. The outer surface of the adapter seat block 10 is also sleeved with a second connecting handle 7, and the second base 9 and the adapter seat block 10, as well as the second connecting handle 7 and the adapter seat block 10 are all snap-fitted. The interior of the second connecting handle 7, the center of the second base 9, and the center of the adapter seat block 10 are all hollow. The end of the second connecting handle 7 away from the handle 5 is fixedly connected and mounted with a suction pipe 11, and the end of the second connecting handle 7 away from the handle 5 is also provided with a visualization component. The second connecting handle 7 is tapered as a whole, with one end close to the handle 5 being larger and the other end being smaller.

The end of the second connecting handle 7 away from the handle 5 is elliptical, the visualization component includes an LED light 12 and a second camera 23 respectively fixedly mounted at two focal points of two ends of the elliptical second connecting handle 7, and the suction pipe 11 is located between the LED light 12 and the second camera 23. The LED light 12 and the second camera 23 are both controlled by the circuit board, and the LED light 12 can provide lighting for the second camera 23.

The end of the second connecting handle 7 away from the handle 5 is in the shape of a rounded rectangle, the visualization component includes a first camera 13 fixedly mounted on the end of the second connecting handle 7 away from the handle 5, and the first camera 13 and the suction pipe 11 are oppositely distributed on the rounded rectangular end face of the second connecting handle 7. The first camera 13 is controlled by the circuit board, and the first camera 13 can capture details inside the user's mouth and display them on the display panel 2.

In the present disclosure, when the location of the tonsil stones is obscured and poses challenges for the operator to locate them, the operator can connect the second connecting handle 7 to handle 5. The terminal end of the second connecting handle 7 is equipped with a first camera 13, which transmits the acquired images to the display panel 2. This functionality enables the operator to observe the condition of the tonsils with greater clarity, thereby facilitating the identification and extraction of the stones.

Furthermore, the LED light 12 and the second camera 23 can be simultaneously used. The LED light 12 serves to illuminate the area for the second camera 23, enhancing the clarity of the images captured, which ultimately aids the operator in the removal of the tonsil stones.

### Embodiment 3

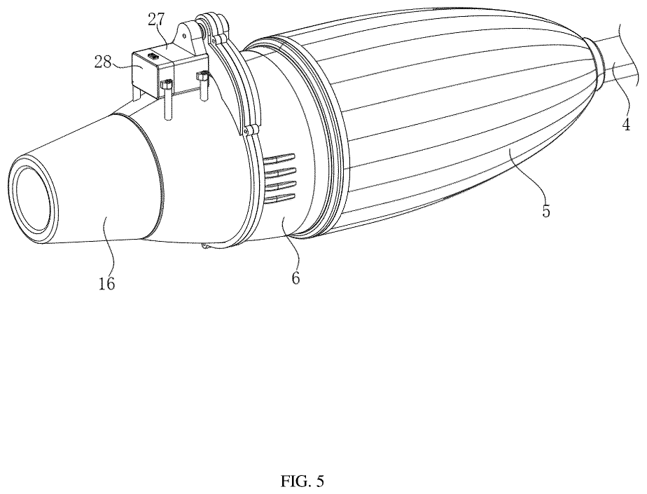

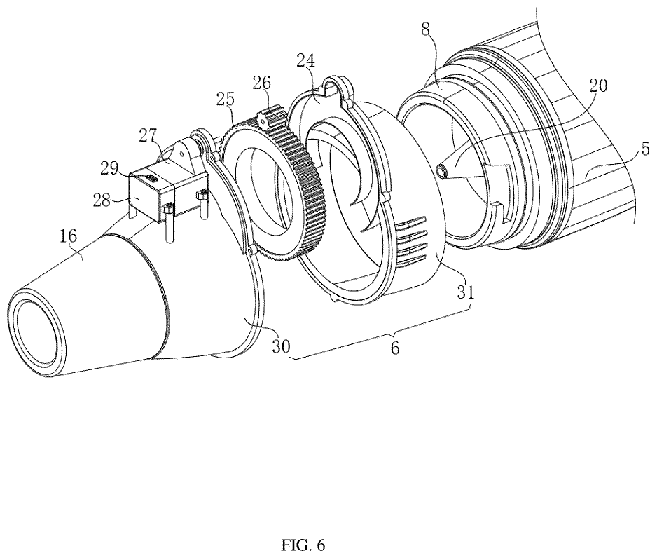

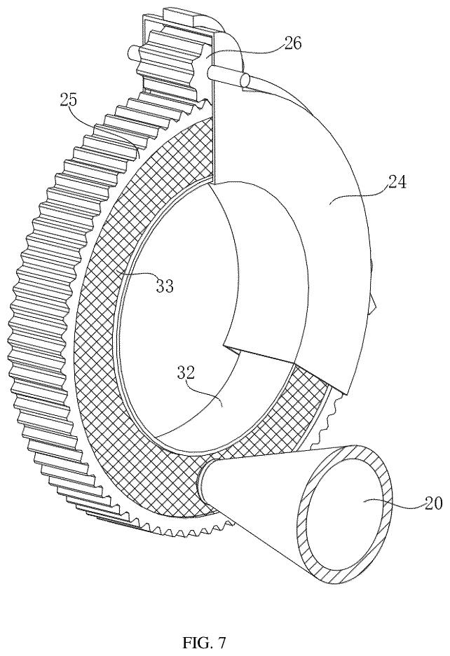

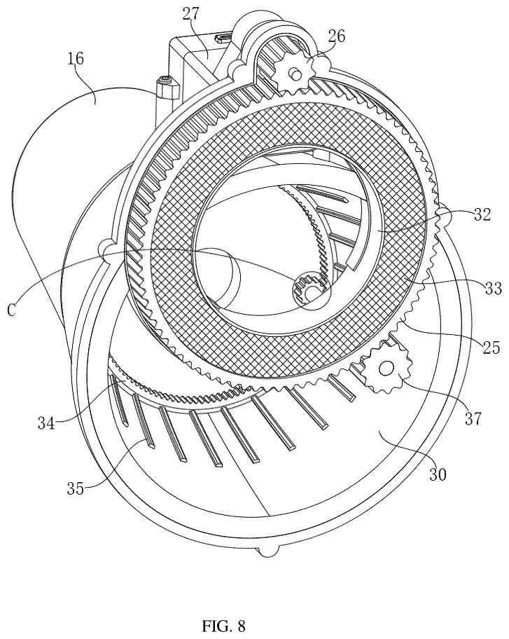

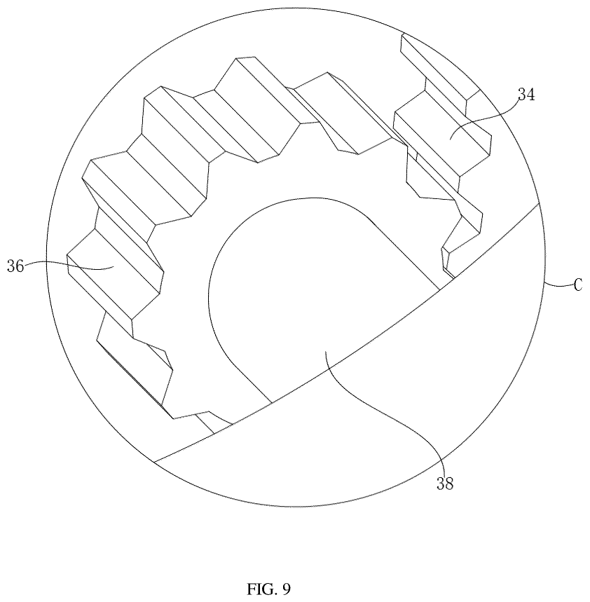

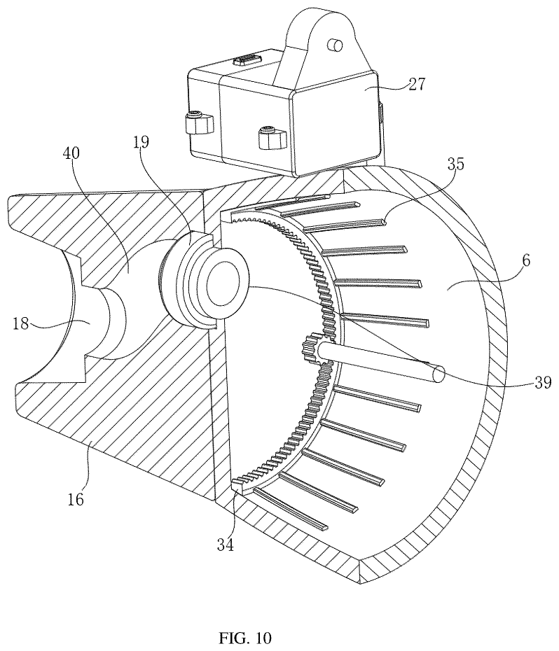

Please refer to FIGS. 5 to 10. This embodiment further illustrates embodiment 1. An end face of the first connecting handle 6 away from the housing is eccentrically arranged with respect to the communicating hole 39, and the suction flow channel 40 is curved. The interval between the eccentrically arranged connecting hole 39 and the bottom of the storage chamber is increased. The entire stone-removal apparatus is tilted downward during operation, and the stones entering the storage chamber will accumulate at the inclined bottom end of the storage chamber.

The protective component includes an eccentric sleeve ring 24 fixedly mounted inside the first connecting handle 6, and the eccentric sleeve ring 24 and the first connecting handle 6 are eccentrically arranged. A portion of the eccentric sleeve ring 24 extending into the first connecting handle 6 is a notch portion. An annular water-absorbing and breathable filter element 33 is provided inside the eccentric sleeve ring 24, and the port of the docking column 20 away from the housing 1 abuts against the surface of the water-absorbing and breathable filter element 33. A driving member is also arranged between the first connecting handle 6 and the eccentric sleeve ring 24, the water-absorbing and breathable filter element 33 has a cotton outer coating with built-in activated carbon particles, which can prevent saliva in the oral cavity from entering the interior of the docking column 20 during suction.

The driving member includes an electric motor 27 fixedly mounted on the outer surface of the first connecting handle 6. The output end of the electric motor 27 is rotatably embedded in the interior of the eccentric sleeve ring 24, and the other end of the electric motor 27 is fixedly connected to a rechargeable power supply 28. The surface of the rechargeable power supply 28 is also provided with a charging port 29 for charging. The inner and outer surfaces of the water-absorbing and breathable filter element 33 are respectively fixedly mounted with a fixing ring 32 and a gear ring 25, and the fixing ring 32 and the gear ring 25 are both slidably mounted in the interior of the eccentric sleeve ring 24. The outer portion of the gear ring 25 is engaged with a first gear 26, and the first gear 26 is axially slidably sleeved on the outer portion of the output end of the electric motor 27. The rechargeable power supply 28 provides power for the electric motor 27 during operation.

The combination structure of the first connecting handle 6 and the eccentric sleeve ring 24 includes a first separation part 30 and a second separation part 31, and the first separation part 30 and the second separation part 31 are assembled with screws, that is, the first separation part 30 and the second separation part 31 can be disassembled and assembled, such that the water-absorbing and breathable filter element 33 can be conveniently replaced.

The electric motor 27 is arranged on the outer surface of the first separation part 30, and the overall volume of the first separation part 30 is less than that of the second separation part 31. Therefore, after the electric motor 27 is assembled, the obstruction of the operator's line of sight can be minimized.

An inner gear ring 34 is rotatably provided at one end face of the interior of the storage chamber away from the water-absorbing and breathable filter element 33. A second gear 36 and a third gear 37 are respectively engaged with the inner wall of the inner gear ring 34 and the outer wall of the gear ring 25, and a rotating shaft 38 is concentrically fixedly connected between the second gear 36 and the third gear 37. The rotating shaft 38 is rotatably mounted to the interior of the storage chamber through a bracket. A plurality of extension strips 35 equidistantly distributed on the circumference of the inner gear ring 34 are fixedly provided at one end face thereof close to the water-absorbing and breathable filter element 33, and each extension strip 35 is slidably fitted with the inner wall of the storage chamber. The inner gear ring 34 can rotate synchronously with the gear ring 25, and the accumulated stones are moved flat by the extension strips 35.

The outer surfaces of the electric motor 27 and the rechargeable power supply 28 are both fixedly covered with a waterproof outer covering to ensure the waterproof performance of the electric motor 27 and the rechargeable power supply 28.

In this disclosure, considering that the suction of saliva occurs concurrently with the negative pressure suction of tonsil stones, to prevent saliva from splashing into the interior of the docking column 20 and causing contamination to the handle 5, the sleeve pipe 4 and the negative pressure suction pump, a preferred solution incorporates a water-absorbing breathable filter element 33, which can be continuously rotated to absorb and process saliva in proximity to the docking column 20, thereby preventing direct entry of saliva into the docking column 20. The rotation of the water-absorbing breathable filter element 33 is facilitated by the engagement and rotation of the first gear 26 and the gear ring 25, which are driven by the electric motor 27.

Furthermore, this disclosure addresses the potential issue of excessive accumulation of stone particles between the storage chamber and the communicating hole 39, which could lead to a backflow of stones into the suction flow channel 40, thereby compromising the suction force of the negative pressure. To counteract this, a preferred solution employs the second gear 36 and the third gear 37, which enable the inner gear ring 34 to rotate in synchrony with the gear ring 25. Additionally, the extension strip 35 is designed to continuously mobilize the accumulated stones, redistributing them along the bottom of the storage chamber to prevent excessive buildup.

### Embodiment 4

Please refer to FIG. 1. This embodiment further illustrates embodiment 2. The connecting member includes a sleeve pipe 4, two ends of which are fixedly assembled with the housing 1 and the handle 5 respectively. An air pipe and a data transmission line are provided inside the sleeve pipe 4, and two ends of the air pipe are connected and docked with the suction end of the negative pressure suction pump and the handle 5 respectively.

In this disclosure, the sleeve pipe 4 may be constructed as either a rigid or flexible pipe. The negative pressure suction pump operates by extracting air from the interior of the handle 5 via the air pipe, thereby creating a negative pressure environment. Additionally, the data transmission line facilitates the real-time display of images captured by either the first camera 13 or the second camera 23 on the display panel 2.

It is important to clarify that in this disclosure, relational terms such as “first,” “second,” and similar designations are employed solely to differentiate one entity or operation from another. These terms do not necessarily indicate or imply any actual relationship or sequence among the entities or operations referenced. Furthermore, the terms “include,” “comprise,” and their variants are intended to denote non-exclusive inclusion. Consequently, a process, method, article, or device that encompasses a series of elements is understood to include not only those specified elements but also additional elements that are not explicitly mentioned, as well as elements that are inherent to the process, method, article, or device in question.

While the embodiments of the present disclosure have been illustrated and described, it is acknowledged by those skilled in the art that various alterations, modifications, substitutions, and variations may be made to these embodiments without deviating from the underlying principles and essence of the present disclosure. The scope of the present disclosure is defined by the appended claims and their equivalents.

1. A separable stone-removal apparatus, comprising a housing, wherein a negative pressure unit is fixedly mounted inside the housing, a display panel for displaying is fixedly mounted on an upper end of the housing, and a circuit board for adjusting an opening and closing state of the negative pressure unit and a display state of the display panel is fixedly arranged inside the housing; an energy storage unit, used to provide power for the circuit board, the negative pressure unit and the display panel, is fixedly arranged inside the housing;
the separable stone-removal apparatus further comprising:
a separable handle unit, suitable for stone removal operations under different working conditions, arranged on an outer side of the housing.

2. The separable stone-removal apparatus according to claim 1, wherein the negative pressure unit is a negative pressure suction pump, the separable handle unit comprises a connecting member mounted inside the housing; the connecting member is mounted with a handle, being hollow inside, on one side thereof away from the housing, and one end of the handle away from the housing is provided with a first suction assembly or a second suction assembly.

3. The separable stone-removal apparatus according to claim 2, wherein the first suction assembly comprises a first base fixedly sleeved on one end of the handle away from the housing, with a center of the first base having an opening; a docking column is fixedly mounted at the opening of the first base; an outer surface of the first base is slidably mounted with a first connecting handle, and the first connecting handle and the first base are snap-fitted; the first connecting handle and an internal space of the first base form a storage chamber for temporary storage of stones; a communicating hole is provided at one end of the first connecting handle away from the handle; a docking component is provided on an end face of the first connecting handle away from the handle, and a protective component is provided inside the storage chamber.

4. The separable stone-removal apparatus according to claim 3, wherein the docking component comprises a first suction head or a second suction head; a suction flow channel is provided inside the first suction head, and a straight groove channel is provided inside the second suction head; an inner diameter of the straight groove channel is less than that of the suction flow channel, and an outer diameter of the second suction head is less than that of the first suction head; a connecting head, which serves to connect the communicating hole with the suction flow channel or the straight groove channel, is fixedly mounted between the first suction head or the second suction head and the communicating hole, and a flaring groove concentrically connected to the suction flow channel is provided at one end of the first suction head away from the first connecting handle.

5. The separable stone-removal apparatus according to claim 3, wherein the protective component comprises an isolation cover sleeved on an outer surface of the docking column; one end of the isolation cover away from the docking column is in a closed shape, and an outer surface of the isolation cover is provided with an air hole for ventilation.

6. The separable stone-removal apparatus according to claim 2, wherein the second suction assembly comprises a second base fixedly arranged on an end face of the handle away from the housing, and another end of the second base is sleeved with an adapter seat block; an outer surface of the adapter seat block is sleeved with a second connecting handle; the second base and the adapter seat block, as well as the second connecting handle and the adapter seat block, are all snap-fitted; an interior of the second connecting handle, a center of the second base and a center of the adapter seat block are all hollow; one end of the second connecting handle away from the handle is fixedly connected and mounted with a suction pipe, and the end of the second connecting handle away from the handle is provided with a visualization component.

7. The separable stone-removal apparatus according to claim 6, wherein the end of the second connecting handle away from the handle is elliptical, and the visualization component comprises an LED light and a second camera respectively fixedly mounted at two focal points of two ends of the elliptical second connecting handle; the suction pipe is located between the LED light and the second camera, and the LED light and the second camera are both controlled by the circuit board.

8. The separable stone-removal apparatus according to claim 6, wherein the end of the second connecting handle away from the handle is rounded rectangular, and the visualization component comprises a first camera fixedly mounted on the end of the second connecting handle away from the handle; the first camera and the suction pipe are oppositely distributed on a rounded rectangular end face of the second connecting handle, and the first camera is controlled by the circuit board.

9. The separable stone-removal apparatus according to claim 4, wherein a straight pipe or an elbow pipe is mounted inside the flaring groove and outside the suction pipe.

10. The separable stone-removal apparatus according to claim 4, wherein the communicating hole and one end face of the first connecting handle away from the housing are eccentrically arranged, and the suction flow channel is in a curved shape.

11. The separable stone-removal apparatus according to claim 3, wherein the protective component comprises an eccentric sleeve ring fixedly mounted inside the first connecting handle, and the eccentric sleeve ring and the first connecting handle are eccentrically arranged; a portion of the eccentric sleeve ring extending into the first connecting handle is a notch portion, and a water-absorbing and breathable filter element, being annular, is provided inside the eccentric sleeve ring; a port of the docking column away from the housing abuts against a surface of the water-absorbing and breathable filter element, and a driving member is provided between the first connecting handle and the eccentric sleeve ring.

12. The separable stone-removal apparatus according to claim 11, wherein the driving member comprises an electric motor fixedly mounted on an outer surface of the first connecting handle; an output end of the electric motor is rotatably embedded inside the eccentric sleeve ring, and another end of the electric motor is fixedly connected to a rechargeable power supply; a surface of the rechargeable power supply is provided with a charging port for charging; an inner and outer surfaces of the water-absorbing and breathable filter element are respectively fixedly sleeved with a fixing ring and a gear ring, and the fixing ring and the gear ring are both slidably mounted inside the eccentric sleeve ring; an outer portion of the gear ring is engaged with a first gear, and the first gear is axially slidably sleeved on an outer portion the output end of the electric motor.

13. The separable stone-removal apparatus according to claim 12, wherein a combination structure of the first connecting handle and the eccentric sleeve ring comprises a first separation part and a second separation part, and the first separation part and the second separation part are assembled by screws.

14. The separable stone-removal apparatus according to claim 13, wherein the electric motor is arranged on an outer surface of the first separation part.

15. The separable stone-removal apparatus according to claim 12, wherein an inner gear ring is rotatably provided at one end face of an interior of the storage chamber away from the water-absorbing and breathable filter element, and a second gear and a third gear are respectively engaged with an inner wall of the inner gear ring and an outer wall of the gear ring; a rotating shaft is concentrically fixedly connected between the second gear and the third gear, and the rotating shaft is rotatably mounted to the interior of the storage chamber through a bracket; a plurality of extension strips are fixedly provided on one end face of the inner gear ring close to the water-absorbing and breathable filter element and are distributed equidistantly around a circumference thereof, and each of the extension strips is slidably fitted with an inner wall of the storage chamber.

16. The separable stone-removal apparatus according to claim 12, wherein an outer surfaces of the electric motor and the rechargeable power supply are fixedly covered with a waterproof outer covering.

17. The separable stone-removal apparatus according to claim 2, wherein the connecting member comprises a sleeve pipe, and two ends of the sleeve pipe are respectively fixedly mounted to the housing and the handle; an air pipe and a data transmission line are provided inside the sleeve pipe, and two ends of the air pipe are respectively connected and docked with a suction end of the negative pressure suction pump and the handle.
